(19) United States
(12) Patent Application Publication
(10) Pub. No.: US 2024/0081661 A1
(43) Pub. Date: Mar. 14, 2024

(54) HEALTH EVALUATION SYSTEM, HEALTH EVALUATION METHOD, AND STORAGE MEDIUM STORING HEALTH EVALUATION PROGRAM

(71) Applicant: HONDA MOTOR CO., LTD., Tokyo (JP)

(72) Inventors: Hideki Sakai, Wako-shi (JP); Shigenobu Mitsuzawa, Wako-shi (JP); Satoru Shinkawa, Wako-shi (JP); Tomohiro Imai, Wako-shi (JP); Keisuke Nakamura, Wako-shi (JP); Hiroshi Ono, Wako-shi (JP)

(21) Appl. No.: 18/460,223

(22) Filed: Sep. 1, 2023

(30) Foreign Application Priority Data
Sep. 14, 2022 (JP) 2022-146524

(51) Int. Cl. A61B 5/021; A61B 5/00

(52) U.S. Cl. CPC A61B 5/021; A61B 5/0004; A61B 5/681; A61B 5/6893; A61B 5/746; A61B 2503/22

(57) ABSTRACT

Provided is a health evaluation system that includes: an acquisition unit that acquires a blood pressure value of a target person; and an evaluation unit that evaluates a health condition of the target person based on the acquired blood pressure value. The evaluation unit evaluates the health condition of the target person based on: a standing-state blood pressure value that is a blood pressure value measured when the target person is heading toward a moving body in a standing state; and a seated-state blood pressure value that is a blood pressure value of the target person measured after boarding the moving body and seated on a seat.

### INCORPORATION BY REFERENCE

The present application claims priority under 35 U.S.C.§ 119 to Japanese Patent Application No. 2022-146524 filed on Sep. 14, 2022. The content of the application is incorporated herein by reference in its entirety.

### BACKGROUND OF THE INVENTION

### Field of the Invention

The present invention relates to a health evaluation system, a health evaluation method, and a non-transitory computer-readable storage medium storing a health evaluation program.

### Description of the Related Art

Japanese Patent Laid-Open No. 2010-57664 discloses a passenger physical condition management device for vehicle in which: the temperature, blood pressure, and the like of a seated passenger is measured at a prescribed time by a sensor provided at a seat or the like of a vehicle and recorded in a database; a health checkup of the passenger onboard the vehicle is conducted; and an alarm is issued or an external report is made when the physical condition of the passenger gets worse.

Japanese Patent Laid-Open No. 2000-279450 discloses a technology with which a seating sensor, a heart rate sensor, a blood pressure monitor, and communication means are provided in a wheel chair, and a report is made to a management sensor when there is an abnormality observed in the heart rate or the blood pressure of a person seated on the wheel chair.

When diagnosing a health condition of a person, there may be cases where it is not possible to make an appropriate diagnosis with only the data of the heart rate and the like in a seated state where the activity of the person is relatively stable.

An object of the present invention is to appropriately diagnose the health condition of a person using a moving body.

### SUMMARY OF THE INVENTION

One aspect of the present invention is a health evaluation system that includes: an acquisition unit that acquires a blood pressure value of a target person; and an evaluation unit that evaluates a health condition of the target person based on the blood pressure value of the target person acquired by the acquisition unit, in which the evaluation unit evaluates the health condition of the target person based on: a standing-state blood pressure value that is a blood pressure value measured when the target person is heading toward a moving body in a standing state; and a seated-state blood pressure value that is a blood pressure value of the target person measured after boarding the moving body and seated on a seat.

According to another aspect of the present invention, the evaluation unit takes, as the standing-state blood pressure value, a blood pressure value acquired by the acquisition unit when the target person is within a predetermined distance range from the moving body.

According to still another aspect of the present invention, the evaluation unit takes: as the standing-state blood pressure value, a blood pressure value acquired immediately after the target person in a standing state boards the moving body and is seated on the seat of the moving body; and as the seated-state blood pressure value, a blood pressure value acquired after a prescribed time defined in advance has passed after the target person is seated on the seat of the moving body.

According to yet another aspect of the present invention, the health evaluation system includes a notification unit that issues a notification based on an evaluation result regarding the health condition of the target person evaluated in the evaluation unit, in which the notification unit determines whether to issue the notification, based on the evaluation result regarding the health condition of the target person.

According to another aspect of the present invention, the health evaluation system includes an information transmission unit that transmits positional information regarding a current position of the moving body to an external server of the moving body at prescribed time intervals, in which the information transmission unit determines whether to transmit the positional information to the server based on the evaluation result regarding the health condition of the target person evaluated in the evaluation unit, or based on whether the notification is issued by the notification unit.

According to still another aspect of the present invention, the acquisition unit acquires the blood pressure value of the target person based on an image of the target person from an inside camera that is provided in the moving body and acquires an image of inside of the moving body.

According to yet another aspect of the present invention, the acquisition unit acquires the blood pressure value of the target person based on an image of the target person from an outside camera that is provided in the moving body and acquires an image of surroundings of the moving body.

According to another aspect of the present invention, the acquisition unit acquires the blood pressure value of the target person by: receiving a blood pressure value measured by a wearable terminal attached to a body of the target person from the wearable terminal; or receiving biological data measured by the wearable terminal, and calculating the blood pressure value based on the received biological data.

According to still another aspect of the present invention, the evaluation unit evaluates the health condition of the target person based on: the standing-state blood pressure value that is the blood pressure value measured when the target person is heading toward the moving body by legged locomotion that is locomotion using own legs in a standing state; and the seated-state blood pressure value that is the blood pressure value of the target person measured after boarding the moving body and seated on the seat.

Another aspect of the present invention is a health evaluation method executed by a computer, and the health evaluation method includes: an acquisition step of acquiring a blood pressure value of a target person; and an evaluation step of evaluating a health condition of the target person based on the blood pressure value of the target person acquired in the acquisition step, in which the evaluation step evaluates the health condition of the target person based on: a standing-state blood pressure value that is a blood pressure value measured when the target person is heading toward the moving body by legged locomotion that is locomotion using own legs in a standing state; and a seated-state blood pressure value that is a blood pressure value of the target person measured after boarding the moving body and seated on a seat.

Still another aspect of the present invention is a health evaluation program causing a computer of a health evaluation system to function as: an acquisition unit that acquires a blood pressure value of a target person; and an evaluation unit that evaluates a health condition of the target person based on the blood pressure value of the target person acquired by the acquisition unit, in which the evaluation unit evaluates the health condition of the target person based on: a standing-state blood pressure value that is a blood pressure value measured when the target person is heading toward a moving body in a standing state; and a seated-state blood pressure value that is a blood pressure value of the target person measured after boarding the moving body and seated on a seat.

According to the present invention, it is possible to appropriately diagnose the health condition of the person using the moving body.

### BRIEF DESCRIPTION OF THE DRAWINGS

FIG. 1 is a diagram showing a configuration of a health evaluation system according to an embodiment of the present invention;

FIG. 2 is a diagram showing an example of a configuration of a vehicle on which an evaluation support device configuring the health evaluation system is loaded, when viewed from the inside;

FIG. 3 is a diagram showing a configuration of a mobile terminal configuring the health evaluation system;

FIG. 4 is a diagram showing a configuration of the evaluation support device configuring the health evaluation system; and

FIG. 5 is a flowchart showing processing procedures of a health evaluation method executed by the health evaluation system.

### DETAILED DESCRIPTION OF THE PREFERRED EMBODIMENTS

### Embodiment

Hereinafter, embodiments of the present invention will be described with reference to the accompanying drawings.

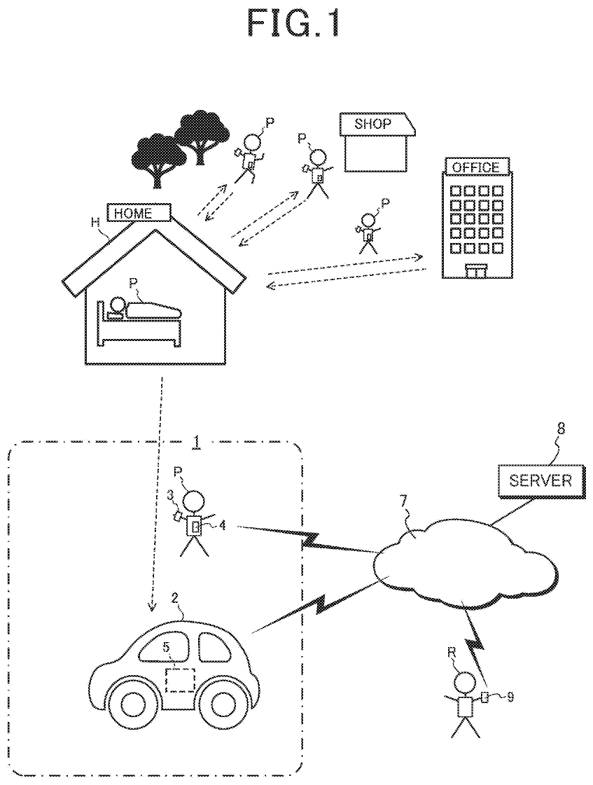

FIG. 1 is a diagram for describing the configuration of a health evaluation system 1 according to one embodiment of the present invention along with an example of a use scene thereof. The health evaluation system 1 evaluates the health condition of a target person P when the target person P is getting into a vehicle 2 as a moving body.

The health evaluation system 1 includes: a wearable terminal 3 and a mobile terminal 4 used by the target person P; and an evaluation support device 5 loaded on the vehicle 2 used by the target person P as a driver. The wearable terminal 3 and the mobile terminal 4, as well as the mobile terminal 4 and the evaluation support device 5 are communicably connected by short range communication such as Bluetooth (registered trademark). Furthermore, the mobile terminal 4 and the evaluation support device 5 are communicably connected to an external server 8 of the vehicle 2 via a communication network 7 such as the Internet. The external server 8 is configured by a computer.

The mobile terminal 4 is a smartphone, for example, and includes a camera, an acceleration sensor, and/or a GNSS (Global Navigation Satellite System) sensor, and the like. The wearable terminal 3 is a wristwatch type terminal device, for example, which, according to a conventional technology, includes various kinds of sensors such as a pulse wave sensor, a heart rate sensor, a blood pressure sensor, an acceleration sensor, and/or a GNSS sensor, and measures biological data of the target person P and transmits the measured biological data (hereinafter, also referred to as biometric data) to the mobile terminal 4.

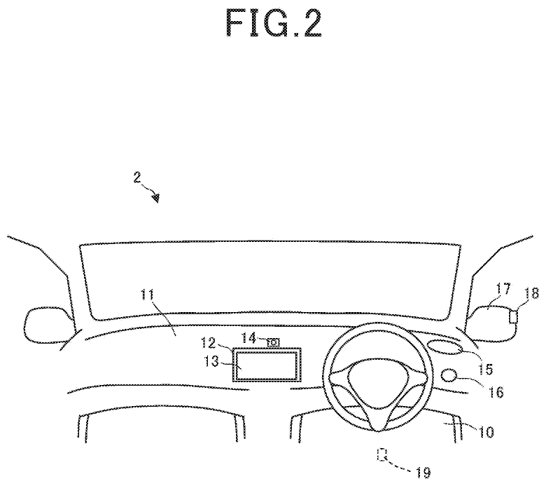

FIG. 2 is a diagram showing an example of the configuration of the vehicle 2 when viewed from the inside. On an instrument panel 11 in front of a driver's seat 10, an AV (Audio-Visual) device 12 and an inside camera 14 are disposed almost in the center in the car width direction. The AV device 12 is a display audio, for example, which displays an image and a video (hereinafter, simply referred to as image) on a first touch panel 13, and acquires information that is input by a touch operation made on the first touch panel 13. Furthermore, the AV device 12 outputs voice and receives voice input by a speaker 15 and a microphone 16 provided in an area of the instrument panel 11 on the driver's seat side. The inside camera 14 is a DMC (Driver Monitoring Camera), for example, and captures images inside the vehicle including images of the driver.

Furthermore, the vehicle 2 includes an outside camera 18 that is provided on a door mirror 17 on a driver's seat 10 side. The outside camera 18 captures images of the external surrounding environment on the right side of the vehicle 2. For example, the outside camera 18 captures images of persons approaching a vehicle door (not shown) on the driver's seat 10 side from the outside on the right side of the vehicle 2. Note that the outside camera 18 may not be limited to a single camera, but may be configured with a plurality of cameras distributed on a car body of the vehicle 2 to capture images of entire or part of the surrounding environment of the vehicle 2.

At the driver's seat 10, a seating sensor 19 for detecting seating of a person is provided. The seating sensor 19 is a pressure sensor that detects the pressure applied to the seat by the weight of the seated person, for example. Note that the seating sensor 19 may be provided not only at the driver's seat 10 but also at all or part of the seats in the vehicle 2 and detect seating of a person on each of the seats.

While a plurality of target persons P are illustrated in FIG. 1, it is to be noted that those indicate various active scenes of the same target person P. The wearable terminal 3 is attached to the arm of the target person P at all times or in most of time zones of the day, and measures biological data of the target person P in various daily activities such as when sleeping, having meals, and the like at home H, exercising (for example, walking, running, body training in a gym), shopping, and commuting to work at prescribed time intervals, for example, consecutively or continuously in time. The wearable terminal 3 transmits the measured biological data to the mobile terminal 4, and the mobile terminal 4 receives and acquires it.

Then, the mobile terminal 4 evaluates the health condition of the target person P based on the biological data acquired from the wearable terminal 3, and issues a notification (hereinafter, also referred to as evaluation notification) based on the evaluation result.

In the present embodiment, specifically, the mobile terminal 4 configuring the health evaluation system 1 evaluates the health condition of the target person P based on the biological data of the target person P when onboard the vehicle 2, that is, the biological data of the target person P in an action until seated on the driver's seat 10 after approaching the vehicle 2 in a standing state.

Specifically, the mobile terminal 4 acquires: a standing-state blood pressure value that is the blood pressure value measured when the target person P is heading toward the vehicle 2 in a standing state; and a seated-state blood pressure value that is the blood pressure value of the target person P measured after boarding the vehicle 2 and seated on the driver's seat 10. Then, the mobile terminal 4 evaluates the health condition of the target person P based on the standing-state blood pressure value and the seated-state blood pressure value. Note here that locomotion of the target person P in the “standing state” may include legged locomotion (for example, traveling by walking or running) performed by using the own legs in a standing state, for example.

In general, those who have large blood pressure fluctuation tend to have a high risk of developing diseases such as stroke and myocardial infarction. With the health evaluation system 1 having the configuration described above, it is possible to accurately acquire the blood pressure value of the target person P in a series of actions when onboard the vehicle 2, and more accurately evaluate the health condition of the target person P boarding the vehicle 2.

Furthermore, since the health evaluation system 1 is capable of accurately evaluating the health condition when the target person P is actually onboard the vehicle 2 as described above, it is possible to support the target person P appropriately in regards to traveling with the vehicle 2 based on the evaluation result. Furthermore, since the health evaluation system 1 acquires the blood pressure value by using the wearable terminal that is attached to the body of the target person P and capable of acquiring the biological data in the immediate vicinity of the body, it is possible to increase the measurement precision of the blood pressure value and evaluate the health condition more accurately. Note, however, that the wearable terminal 3 is merely an example of means for acquiring the standing-state blood pressure value and the seated-state blood pressure value, and the health evaluation system 1 may acquire those blood pressure values from other sensing devices such as a camera, for example.

Evaluation of the health condition is conducted by calculating an evaluation score (points) indicating whether the health condition is good or poor, for example, based on the scoring criteria regarding the standing-state blood pressure value, the seated-state blood pressure value, and a difference between the standing-state blood pressure value and the seated-state blood pressure value. The evaluation score may indicate higher points as the health condition is finer, for example. The reference criteria may also be defined in advance.

Furthermore, the mobile terminal 4 determines whether an evaluation notification based on the evaluation result is to be issued based on the evaluation result regarding the health condition of the target person P. For example, the mobile terminal 4 may issue an evaluation notification when the evaluation score is lower than a prescribed threshold (that is, when the health condition is lower than a prescribed level). The evaluation notification may be a notification for giving an alert, suggesting alternative means, or the like for the target person P in the vehicle 2, for example, such as “Your health condition is not well, so please drive carefully”, “Your health condition is not good, so please do not drive today and take train or taxi for transportation”, or the like. The mobile terminal 4 gives an instruction to the evaluation support device 5 of the vehicle 2, for example, and outputs a text message or a voice message from the first touch panel 13 and/or the speaker 15 loaded on the vehicle to issue the evaluation notification.

Instead of or in addition to that, the evaluation notification may be a notification transmitted for reporting the circumstance such as “xxx (target person P) seems to be in poor health condition, but is about to start driving a vehicle” from the mobile terminal 4 to a mobile terminal 9 (for example, smartphone) of a related person R such as a family member or a friend of the target person P.

Thereby, with the health evaluation system 1, an evaluation notification regarding the health condition is issued only when the health condition is not well, for example, based on the evaluation result of the health condition. Thus, it is possible to avoid causing a trouble to the target person P or the related person R as the notification recipient by issuing an unnecessary notification regarding the health condition.

Furthermore, with the health evaluation system 1, by issuing a notification to the target person P when the health condition is not well, for example, the target person P can objectively grasp the own health condition at an appropriate timing. Therefore, it is possible with the health evaluation system 1 to encourage the target person P to make an appropriate determination regarding traveling by a moving body.

Furthermore, based on the evaluation result regarding the health condition of the target person P and/or based on the fact that the evaluation notification is issued, the mobile terminal 4 gives an instruction to the evaluation support device 5 of the vehicle 2 to transmit positional information of the vehicle 2 to the server 8 outside of the vehicle 2 at prescribed time intervals. Thereby, with the health evaluation system 1, when the health condition of the target person P is evaluated as poor or when a notification is issued regarding the health condition, the related person R of the target person P such as a family member or a friend can trace the whereabouts and traveling route of the target person P by accessing the server 8 using the own mobile terminal 9. Therefore, it is possible to keep an eye on the movement of the target person P.

Instead of giving an instruction to the evaluation support device 5 to transmit the positional information of the vehicle 2 to the server 8, the mobile terminal 4 may receive the positional information of the vehicle 2 from the evaluation support device 5 at prescribed time intervals and transmit it to the server 8.

Hereinafter, the configurations of the mobile terminal 4 and the evaluation support device 5 will be described.

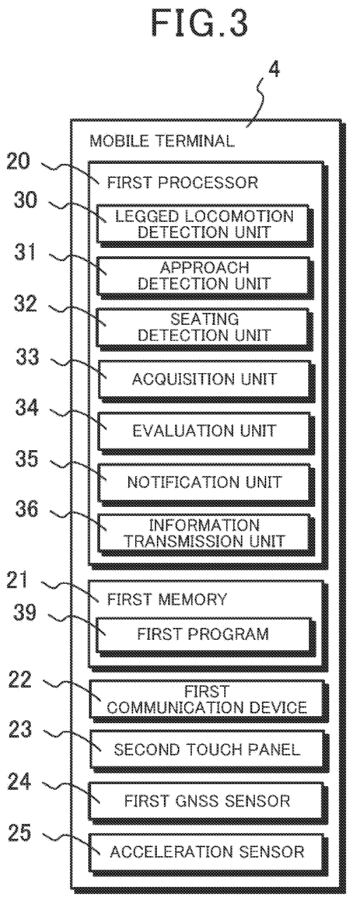

FIG. 3 is a diagram showing the configuration of the mobile terminal 4 configuring the health evaluation system 1.

The mobile terminal 4 is a smartphone, for example. The mobile terminal 4 includes a first processor 20, a first memory 21, a first communication device 22, a second touch panel 23, a first GNSS sensor 24, and an acceleration sensor 25. The acceleration sensor 25 is a nine-axis acceleration sensor, for example.

The first memory 21 may be configured by a volatile or nonvolatile semiconductor memory. The first communication device 22 is a transceiver for allowing the mobile terminal 4 to communicate with the wearable terminal 3, the evaluation support device 5, and/or the server 8. For example, the first communication device 22 may include: a short range communication device for performing short range communication complying with Bluetooth (registered trademark) communication standards with the wearable terminal 3 and the evaluation support device 5 of the vehicle 2; and a long range communication device for communicating with the server 8 via the communication network 7.

The first processor 20 is a computer that includes a CPU (Central Processing Unit) and the like, for example. The first processor 20 may include a ROM (Read Only Memory) where programs are loaded, a RAM (Random Access memory) for temporarily storing data, and the like. Furthermore, the first processor 20 includes, as functional elements or functional units, a legged locomotion detection unit 30, an approach detection unit 31, a seating detection unit 32, an acquisition unit 33, an evaluation unit 34, a notification unit 35, and an information transmission unit 36.

Those functional elements of the first processor 20 are implemented, for example, by executing a first program 39 stored in the first memory 21 by the first processor 20 that is a computer. The first program 39 may be stored in any computer readable storage media. Instead, all or part of the functional elements of the first processor 20 may be configured with hardware each including one or more electronic circuit components. Note here that the first program 39 together with a second program 69 executed in the evaluation support device 5 configures a health evaluation program of the present disclosure.

The legged locomotion detection unit 30 detects that the target person P is conducting a legged locomotion. Note here that “legged locomotion” means locomotion of a person conducted by using own legs in a standing state, such as traveling by standing and walking or running, for example. The legged locomotion detection unit 30, according to a conventional technology, counts the number of steps of the target person P based on a sensor signal from the acceleration sensor 25 of the mobile terminal 4, and determines that the target person P is conducting legged locomotion while the number of steps is being counted up. Furthermore, the legged locomotion detection unit 30 determines that the target person P is no longer conducting legged locomotion when the number of steps is no longer counted up.

The approach detection unit 31 detects that the target person P is within a prescribed first distance range (for example, within 10 meters) defined in advance from the vehicle 2. Furthermore, when the target person P is within the first distance range, the approach detection unit 31 detects the traveling direction of the target person P and determines whether the target person P is heading toward the vehicle 2.

For example, the approach detection unit 31 detects that the target person P enters the first distance range from the vehicle 2 and that the target person P is heading toward the vehicle 2 based on a received radio wave intensity and chronological change thereof in the Bluetooth communication performed between the first communication device 22 and the evaluation support device 5 of the vehicle 2.

Specifically, the approach detection unit 31 searches a Bluetooth device existing in the surroundings of the mobile terminal 4 by the first communication device 22 and, when Bluetooth communication is established with the evaluation support device 5 of the vehicle 2, calculates the distance between the target person P who is carrying the mobile terminal 4 and the vehicle 2 based on the received radio wave intensity of the Bluetooth communication the first communication device 22 receives from the evaluation support device 5. The approach detection unit 31 determines whether the target person P has entered the first distance range from the vehicle 2 based on the value of the calculated distance. Furthermore, the approach detection unit 31 calculates the distance at prescribed time intervals, and determines whether the target person P is heading toward the vehicle 2.

Instead, the approach detection unit 31 may acquire an image of the surroundings of the vehicle 2 captured by the outside camera 18 of the vehicle 2 from the evaluation support device 5 when Bluetooth communication is established with the evaluation support device 5 of the vehicle 2, and detect that the target person P is approaching the vehicle 2 based on the acquired image.

The seating detection unit 32 detects that the target person P is seated on a seat of the vehicle 2. The seat to be the target of seating detection in the present embodiment is the driver's seat 10, for example. Note, however, that the seat to be the target of seating detection is not limited to the driver's seat 10 but may also be any other seats in the vehicle 2.

Specifically, the seating detection unit 32 detects that the target person P is seated on the seat of the vehicle 2 by acquiring, from the evaluation support device 5, seating information that is the information indicating whether a person is seated on the driver's seat 10 by having the Bluetooth communication with the evaluation support device 5 via the first communication device 22.

For example, the seating detection unit 32 first determines whether the mobile terminal 4 is in the immediate vicinity of the vehicle 2, based on the distance between the mobile terminal 4 and the vehicle 2 measured from the received radio wave intensity of the Bluetooth communication. More specifically, the seating detection unit 32, for example, determines whether the mobile terminal 4 is within a prescribed second distance range (for example, within 1 meter) from the vehicle 2. Then, when the mobile terminal 4 is within the second distance range from the vehicle 2, the seating detection unit 32 repeatedly acquires seating information from the evaluation support device 5, and determines that the target person P is seated on the driver's seat 10 at the time where the seating information indicating that a person is seated on the driver's seat 10 is received for the first time (hereinafter, also referred to as seated time).

Instead, the seating detection unit 32 may acquire an image captured inside the vehicle 2 by the inside camera 14 of the vehicle 2 from the evaluation support device 5 when Bluetooth communication is established with the evaluation support device 5 of the vehicle 2, and detect that the target person P is seated on the driver's seat 10 based on the acquired image of the inside. The seating detection unit 32 can take, as the seated time, the time at which it is detected based on the image that the target person P is seated on the driver's seat 10.

Recognition of a face image of the target person P in the surrounding image captured by the outside camera 18 and the inside image captured by the inside camera 14 may be performed by storing in advance a reference face image of the target person P for reference in the first memory 21, for example, and performing matching processing with the reference face image according to a conventional technology.

The acquisition unit 33 acquires the blood pressure value of the target person P. For example, the acquisition unit 33 continuously receives and acquires, from the wearable terminal 3, the biological data of the target person P measured at prescribed time intervals by the wearable terminal 3 the target person P is wearing. When the blood pressure value is included in the biological data received from the wearable terminal 3, the acquisition unit 33 acquires the blood pressure value by receiving the biological data. Alternatively, when the blood pressure value is not included in the biological data received from the wearable terminal 3, the acquisition unit 33 may calculate and acquire the blood pressure value based on the received biological data. For example, the acquisition unit 33 can calculate the blood pressure value according to a conventional technology based on the pulse wave or the like included in the biological data.

The evaluation unit 34 evaluates the health condition of the target person P form the blood pressure value of the target person P acquired by the acquisition unit 33. In the present embodiment, specifically, the evaluation unit 34 evaluates the health condition of the target person P based on the standing-state blood pressure value that is the blood pressure value measured when the target person P is heading toward the vehicle 2 in a standing state, and the seated-state blood pressure value that is the blood pressure value of the target person P measured after boarding the vehicle 2 and seated on the seat.

As described above, those who have large blood pressure fluctuation tend to have a high risk of developing diseases such as stroke and myocardial infarction. With the health evaluation system 1 having the configuration described above, it is possible to accurately acquire the blood pressure value of the target person P from before and after boarding the vehicle 2, so that the health condition of the target person P boarding the vehicle 2 can be evaluated more accurately.

Note here that locomotion of the target person P in the “standing state” includes legged locomotion (for example, traveling by walking or running) performed by using the own legs in a standing state, for example. The evaluation unit 34 takes, as the standing-state blood pressure value, the blood pressure value that is acquired by the acquisition unit 33 while the legged locomotion detection unit 30 is detecting legged locomotion of the target person P and the approach detection unit 31 is detecting approaching of the target person P toward the vehicle 2 within the first distance range from the vehicle 2. When the blood pressure value is acquired for a plurality of times while the target person P is approaching the vehicle 2 within the first distance range, the evaluation unit 34 may take, as the standing-state blood pressure value, statistics such as the maximum, minimum, mean, or median of the prescribed number of blood pressure values acquired immediately before, for example.

Furthermore, when the seating detection unit 32 detects that the target person P is seated on the driver's seat 10, the evaluation unit 34 takes the blood pressure value acquired by the acquisition unit 33 after the seated time as the seated-state blood pressure value. For example, the evaluation unit 34 can take, as the seated-state blood pressure value, statistics such as the maximum, minimum, mean, or median of the blood pressure values acquired for a plurality of times after the seated time.

Instead, the evaluation unit 34 may take, as the standing-state blood pressure value, the blood pressure value acquired by the acquisition unit 33 at the seated time or immediately after the seated time. Furthermore, the evaluation unit 34 may take, as the seated-state blood pressure value, the blood pressure value (or the statistics) acquired after a prescribed time defined in advance has passed after the target person P is seated on the driver's seat 10 of the vehicle 2. This makes it possible to more appropriately evaluate the health condition of the target person P at the point of boarding the vehicle 2.

Then, the evaluation unit 34 evaluates the health condition of the target person P based on the standing-state blood pressure value and the seated-state blood pressure value. As described above, the evaluation is made by calculating the evaluation score indicating whether the health condition is well or not based on the scoring criteria defined in advance regarding the standing-state blood pressure value, the seated-state blood pressure value, and the difference between the standing-state blood pressure value and the seated-state blood pressure value. The evaluation score is expressed by the points ranged from 0 to 100, for example, and may indicate that the higher the point, the finer the health condition.

Note here that the health condition can be divided into any number of levels by the range of the points of the evaluation scores. In the present embodiment, as an example, the health condition is divided into a first level that is “good” condition where the evaluation score is 70 or higher, a second level that is a “caution-required” condition where the evaluation score is 40 or higher and less than 70, and a third level that is a “poor” condition where the evaluation score is less than 40.

Referring to FIG. 3, the notification unit 35 issues a notification (hereinafter, also referred to as evaluation notification) based on the evaluation result of the health condition of the target person P acquired by the evaluation unit 34. As described above, the evaluation notification may be a notification for giving an alert, suggesting alternative means, or the like for the target person P in the vehicle 2 (in the present embodiment, the target person P seated on the driver's seat 10 of the vehicle 2 as the driver). In the present embodiment, the notification unit 35 issues the evaluation notification by giving an instruction to the evaluation support device 5 of the vehicle 2 to output a text message or a voice message from the first touch panel 13 or the speaker 15 loaded on the vehicle.

Alternatively, as described above, the evaluation notification may be a notification for reporting the circumstance including the information about the health condition of the target person P, which is transmitted to the mobile terminal 9 such as a smartphone of the related person R such as a family member or a friend of the target person P. Access information (e-mail address or the like) for the mobile terminal 9 of the related person R to be notified may be stored in advance in the first memory 21 of the mobile terminal 4 as notification recipient information.

In the present embodiment, the notification unit 35 determines whether to issue the evaluation notification based on the evaluation result regarding the health condition of the target person P acquired by the evaluation unit 34. For example, the notification unit 35 may not issue the evaluation notification when the health condition of the target person P indicated in the evaluation result is the first level (good condition), and issue the evaluation notification when the health condition is the second level or worse (that is, the second level (caution-required condition) or the third level (poor condition)).

Note that the content of the evaluation notification may vary depending on the evaluation result regarding the health condition. For example, the notification unit 35 may issue the evaluation notification for giving an alert as described above when the health condition is the second level, and may issue the notification for suggesting alternative means or reporting the circumstance when the health condition is the third level.

The information transmission unit 36 transmits the positional information of the current position of the vehicle 2 to the server 8 outside the vehicle 2 at prescribed time intervals. The positional information may be transmitted by a positional information transmission unit 64 (to be described later) of the evaluation support device 5 by giving an instruction from the information transmission unit 36 to the evaluation support device 5 of the vehicle 2. Alternatively, the information transmission unit 36 may receive the positional information of the vehicle 2 from the evaluation support device 5 at prescribed time intervals or may acquire the positional information from the first GNSS sensor 24 of the mobile terminal 4 at prescribed time intervals, and transmit it to the server 8.

In the present embodiment, specifically, the information transmission unit 36 determines whether to transmit the positional information based on the evaluation result regarding the health condition of the target person P acquired by the evaluation unit 34 or based on whether the evaluation notification is issued by the notification unit 35. For example, the information transmission unit 36 may transmit the positional information, when the evaluation result of the health condition acquired by the evaluation unit 34 is the third level or when an evaluation notification is issued by the notification unit 35.

Thereby, for example, the related person R of the target person P such as a family member or a friend can trace the whereabouts and traveling route of the target person P by accessing the server 8 using the own mobile terminal 9. Therefore, it is possible to keep an eye on the movement of the target person P, when the health condition of the target person P is not well, or the like. Furthermore, whether to transmit the positional information is determined based on the evaluation result or whether the evaluation notification is issued, so that the privacy of the target person P can be protected as usual in an ordinal case where the health condition is good, for example.

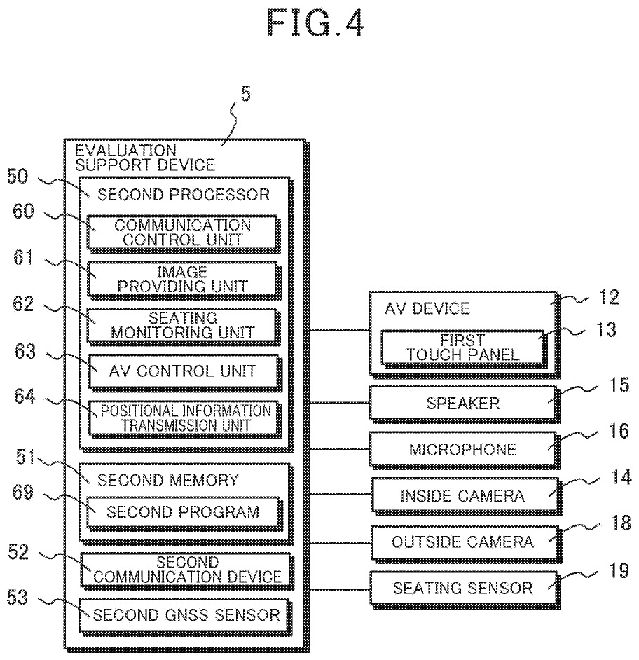

FIG. 4 is a diagram showing the configuration of the evaluation support device 5 provided in the vehicle 2.

The evaluation support device 5 includes a second processor 50, a second memory 51, a second communication device 52, and a second GNSS sensor 53.

The second memory 51 may be configured by a volatile or nonvolatile semiconductor memory. The second communication device 52 is a transceiver for allowing the evaluation support device 5 to communicate with the mobile terminal 4, the server 8 and/or other in-vehicle devices of the vehicle 2. For example, the second communication device 52 may include: a short range communication device for performing short range communication complying with Bluetooth (registered trademark) communication standards with the mobile terminal 4; a long range communication device for communicating with the server 8 via the communication network 7; and a network communication device for performing communication with various kinds of in-vehicle electronic control devices that perform operation control and operation monitoring of the vehicle 2 via an in-vehicle network.

The second processor 50 is a computer that includes a CPU and the like, for example. The second processor 50 may include a ROM where programs are loaded, a RAM for temporarily storing data, and the like. Furthermore, the second processor 50 includes, as functional elements or functional units, a communication control unit 60, an image providing unit 61, a seating monitoring unit 62, an AV control unit 63, and a positional information transmission unit 64.

Those functional elements of the second processor 50 are implemented, for example, by executing the second program 69 stored in the second memory 51 by the second processor 50 that is a computer. The second program 69 may be stored in any computer readable storage media. Instead, all or part of the functional elements of the second processor 50 may be configured with hardware each including one or more electronic circuit components. As described above, the second program 69 together with the first program 39 executed in the mobile terminal 4 configures the health evaluation program of the present disclosure.

The communication control unit 60 performs control and monitoring of the communication operations of the second communication device 52. In the present embodiment, specifically, the communication control unit 60 detects that the second communication device 52 has established connection of the Bluetooth communication with the mobile terminal 4.

In response to a request received from the mobile terminal 4, the image providing unit 61 transmits, to the mobile terminal 4, an image of the inside of the vehicle 2 captured by the inside camera 14 and/or an image of the surroundings of the vehicle 2 captured by the outside camera 18.

The seating monitoring unit 62 monitors whether a person has seated on a seat of the vehicle 2. Furthermore, in response to a request received from the mobile terminal 4, the seating monitoring unit 62 transmits, to the mobile terminal 4, seating information that is the information indicating whether a person is being seated. In the present embodiment, the seating monitoring unit 62 monitors whether a person is seated on the driver's seat 10 by the seating sensor 19 provided at the driver's seat 10. Instead, the seating monitoring unit 62 may monitor seating on the driver's seat 10 based on the image of the inside of the vehicle 2 captured by the inside camera 14.

The AV control unit 63 outputs, by the AV device 12, the image information and the voice information using the first touch panel 13 and the speaker 15. Furthermore, the AV control unit 63 receives and processes operation input from the first touch panel 13 and voice input from the microphone 16. In the present embodiment, specifically, the AV control unit 63 outputs the evaluation notification for the target person P described above by the AV device 12 according to an instruction received from the mobile terminal 4. The output may be image output such as text via the first touch panel 13 and/or may be voice output via the speaker 15.

In response to an instruction received from the mobile terminal 4, the positional information transmission unit 64 acquires the positional information indicating the current position of the vehicle 2 from the second GNSS sensor 53 at prescribed time intervals, and transmits it to the server 8. Furthermore, in response to a request received from the mobile terminal 4, the positional information transmission unit 64 transmits the positional information indicating the current position of the vehicle 2 to the mobile terminal 4.

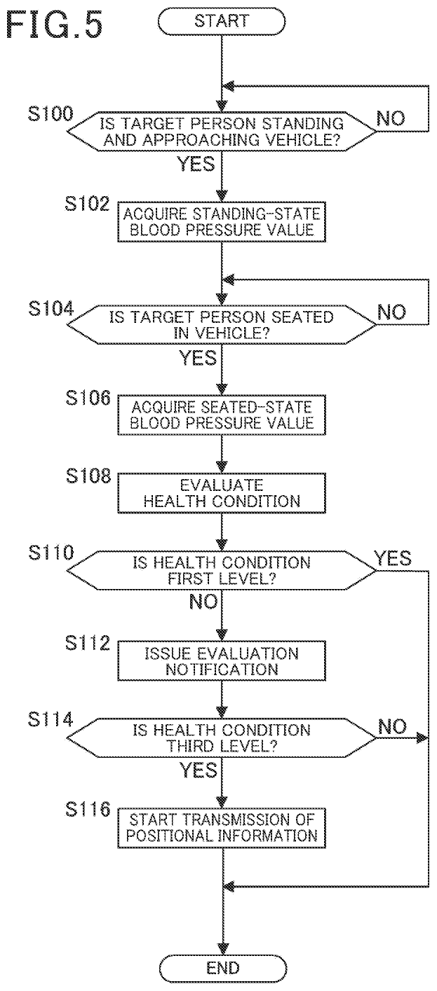

Next, operation procedures performed in the health evaluation system 1 will be described. FIG. 5 is a flowchart showing the processing procedures of a health evaluation method executed by the health evaluation system 1. The processing of FIG. 5 is executed repeatedly.

When the processing is started, first, the approach detection unit 31 of the mobile terminal 4 configuring the health evaluation system 1 determines whether the target person P carrying the mobile terminal 4 is close to the first distance range from the vehicle 2 by legged locomotion (that is, in a standing state) based on the information and the like from the legged locomotion detection unit 30 (S100). Then, when the target person P is not close to the vehicle 2 (NO in S100), the approach detection unit 31 returns to Step S100 to repeat the processing, and waits for the target person P to approach the vehicle 2.

On the other hand, when the target person P is close to the vehicle 2 (YES in S100), the evaluation unit 34 acquires, as the standing-state blood pressure value, the blood pressure value of the target person P acquired by the acquisition unit 33 (S102). Then, the seating detection unit 32 determines whether the target person P is seated on a seat of the vehicle 2 (S104). When the target person P is not seated (NO in S104), the seating detection unit 32 returns to Step S104 to repeat the processing, and waits for the target person P to be seated.

On the other hand, when the target person P is seated (YES in S104), the evaluation unit 34 acquires, as the seated-state blood pressure value, the blood pressure value of the target person P acquired by the acquisition unit 33 (S106). Then, the evaluation unit 34 evaluates the health condition of the target person P based on the acquired standing-state blood pressure value and seated-state blood pressure value (S108).

Subsequently, the notification unit 35 determines whether to issue a notification based on the evaluation result acquired in Step S108. For example, when the level of the health condition of the target person P indicated in the evaluation result acquired in Step S108 is a prescribed level, the notification unit 35 determines to issue a notification. Specifically, for example, the notification unit 35 determines whether the evaluation result acquired in Step S108 indicates that the health condition of the target person P is the first level described above (S110).

Then, when the health condition of the target person P is the second level or the third level, that is, when not the first level (NO in S110), the notification unit 35 issues the evaluation notification described above (S112). As described above, the evaluation notification may be issued for the target person P by the AV control unit 63 of the evaluation support device 5 by an instruction from the notification unit 35, and/or by transmitting a notification to the mobile terminal 9 of the related person R such as a family member or a friend from the notification unit 35.

Then, the information transmission unit 36 determines whether to transmit the positional information of the vehicle 2 to the server 8 based on the evaluation result acquired in Step S108. For example, when the level of the health condition of the target person P indicated in the evaluation result acquired in Step S108 is a prescribed level, the notification unit 35 determines to issue a notification. Specifically, for example, the notification unit 35 determines whether the evaluation result acquired in Step S108 indicates that the health condition of the target person P is the third level described above (S114).

Then, when the health condition of the target person P is the third level described above (YES in Step S114), the information transmission unit 36 starts transmission of the positional information of the vehicle 2 to the server 8 (S116) and ends the processing. As described above, transmission of the positional information may be executed at prescribed time intervals by the positional information transmission unit 64 of the evaluation support device 5 by an instruction given to the evaluation support device 5 from the information transmission unit 36. On the other hand, when the health condition of the target person P is not the third level described above (that is, the second level) (NO in Step S114), the information transmission unit 36 ends the processing without transmitting the positional information of the vehicle 2.

Furthermore, when the health condition of the target person P is determined as the first level in Step S110 (YES in S110), the notification unit 35 ends the present processing.

As described above, the information transmission unit 36 may determine whether to start transmission of the positional information based on whether the evaluation notification is issued by the notification unit 35. In that case, in Step S114, whether the evaluation notification is issued may be determined instead of determining whether the evaluation result of the health condition indicates to be in the third level.

In the above, Step S100 to Step S106 correspond to an acquisition step of the present disclosure. Furthermore, Step S108 corresponds to an evaluation step of the present disclosure.

### Another Embodiment

While the target person P in the embodiment above is described to be a person using the vehicle 2 as the driver, the target person P is not limited to a driver. The target person P may be any persons onboard using the vehicle 2 (for example, fellow passengers).

Furthermore, while the vehicle 2 is described as an example of a moving body in the embodiment above, the moving body where the target person P is onboard is not limited to a vehicle but may also be any moving body such as a ship or an airplane where the target person P can be seated and boarded.

Furthermore, while the acquisition unit 33 in the embodiment above is described to acquire the blood pressure value from the biological data acquired by the wearable terminal 3 attached to the body of the target person P, acquisition means of the blood pressure value is not limited thereto. For example, the acquisition unit 33 may acquire the blood pressure value of the target person P according to a conventional technology (for example, see Japanese Patent Laid-Open No. 2022-008806), based on an image of the target person P from the inside camera 14 that is provided in the vehicle 2 and acquires the images on the inner side of the vehicle 2. Thereby, even when the target person P is not wearing a device such as the wearable terminal 3, it is possible to have any person inside the vehicle 2 as the target person P and acquire the blood pressure value of that target person. Note that the acquisition unit 33 can acquire the image inside the vehicle captured by the inside camera 14 by Bluetooth communication via the evaluation support device 5. Furthermore, the acquisition unit 33 may, according to the conventional technology, extract an image of the target person P as a specific person from an inside image using face image recognition processing such as matching processing described above, for example, and acquire the blood pressure value regarding the target person P.

Furthermore, the acquisition unit 33 may acquire the blood pressure value of the target person P based on an image of the target person P from the outside camera 18 that is provided on the vehicle 2 and acquires images in the surroundings of the vehicle 2. Thereby, it is possible to have any person in the surroundings of the vehicle 2 as the target person P and acquire the blood pressure value of that target person P without having the target person P wear a device such as the wearable terminal 3. Note that, as in the case described above, the acquisition unit 33 can acquire the surrounding image captured by the outside camera 18 by Bluetooth communication via the evaluation support device 5. Furthermore, specification of a person from the surrounding image and acquisition of the blood pressure value from the surrounding image can be performed in the same manner as the case described above.

Furthermore, part of the functional elements of the mobile terminal 4 according to the present embodiment may be distributed to other devices such as the evaluation support device 5 that is connected to the mobile terminal 4 in a communicable manner. For example, the evaluation unit 34, the notification unit 35, and/or the information transmission unit 36 provided in the mobile terminal 4 illustrated in FIG. 3 may be provided in the evaluation support device 5.

Alternatively, the health evaluation system 1 may include an additional server for image processing, which is connected to the mobile terminal 4 and the evaluation support device 5 in a communicable manner, and the evaluation unit 34, the notification unit 35, and/or the information transmission unit 36 may be implemented by a computer on the additional server. Alternatively, the evaluation unit 34, the notification unit 35, and/or the information transmission unit 36 may be implemented by being distributed to the mobile terminal 4, the evaluation support device 5, and the additional server. In those cases, the first program 39 that is executed by the mobile terminal 4, the second program 69 that is executed by the evaluation support device 5, and a program that is executed by the computer of the additional server as a whole correspond to a health evaluation program of the present disclosure.

Note that the present invention is not limited to the configurations of the embodiments described above, but may also be implemented in various modes without departing from the scope of the appended claims.

### Configurations Supported by the Embodiments

The above-described embodiments support the following items.

(Item 1) A health evaluation system including: an acquisition unit that acquires a blood pressure value of a target person; and an evaluation unit that evaluates a health condition of the target person based on the blood pressure value of the target person acquired by the acquisition unit, in which the evaluation unit evaluates the health condition of the target person based on: a standing-state blood pressure value that is a blood pressure value measured when the target person is heading toward a moving body in a standing state; and a seated-state blood pressure value that is a blood pressure value of the target person measured after boarding the moving body and seated on a seat.

According to the health evaluation system of item 1, the health condition of the target person is evaluated based on the blood pressure value under a standing state measured when the target person is actually onboard the moving body and the blood pressure value under a seated state, so that it is possible to appropriately support the target person in regards to traveling in the moving body based on the evaluation result.

(Item 2) The health evaluation system according to item 1, in which the evaluation unit takes, as the standing-state blood pressure value, a blood pressure value acquired by the acquisition unit when the target person is within a predetermined distance range from the moving body.

According to the health evaluation system of item 2, the blood pressure value of the target person from immediately before boarding the moving body till being onboard can be accurately acquired, so that it is possible to evaluate the health condition of the target person boarding the moving body more accurately.

(Item 3) The health evaluation system according to item 1 or 2, in which the evaluation unit takes: as the standing-state blood pressure value, a blood pressure value acquired immediately after the target person in a standing state boards the moving body and is seated on the seat of the moving body; and as the seated-state blood pressure value, a blood pressure value acquired after a prescribed time defined in advance has passed after the target person is seated on the seat of the moving body.

According to the health evaluation system of item 3, the blood pressure value acquired immediately after being seated on the seat of the moving body is equivalently measured as the standing-state blood pressure value acquired immediately before being seated, and also the stable blood pressure value acquired after a prescribed time has passed after being seated is taken as the seated-state blood pressure value. Therefore, the health condition of the target person at the point of boarding the moving body can be evaluated more appropriately.

(Item 4) The health evaluation system according to any one of items 1 to 3, including a notification unit that issues a notification based on an evaluation result regarding the health condition of the target person evaluated in the evaluation unit, in which the notification unit determines whether to issue the notification, based on the evaluation result regarding the health condition of the target person.

According to the health evaluation system of item 4, a notification regarding the health condition is issued only when the health condition is not well, for example, based on the evaluation result of the health condition, so that it is possible to avoid causing a trouble to the target person or the related person as the notification recipient by issuing an unnecessary notification regarding the health condition. Furthermore, according to the health evaluation system of item 4, by issuing a notification to the target person when the health condition is not well, for example, the target person can objectively grasp the own health condition at an appropriate timing. Therefore, it is possible with the health evaluation system of item 4 to encourage the target person to make an appropriate determination regarding traveling by the moving body.

(Item 5) The health evaluation system according to item 4, including an information transmission unit that transmits positional information regarding a current position of the moving body to an external server of the moving body at prescribed time intervals, in which the information transmission unit determines whether to transmit the positional information to the server based on the evaluation result regarding the health condition of the target person evaluated in the evaluation unit, or based on whether the notification is issued by the notification unit.

According to the health evaluation system of item 5, when the health condition is evaluated as poor or when a notification is issued regarding the health condition, the related person of the target person such as a family member or a friend can trace the whereabouts and traveling route of the target person by accessing the server using the own mobile terminal. Therefore, it is possible to keep an eye on the movement of the target person.

(Item 6) The health evaluation system according to any one of items 1 to 5, in which the acquisition unit acquires the blood pressure value of the target person based on an image of the target person from an inside camera that is provided in the moving body and acquires an image of inside of the moving body.

According to the health evaluation system of item 6, the blood pressure value of the target person is acquired based on the image of the inside of the moving body, so that it is possible to have any person inside the moving body as the target person and acquire the blood pressure value of the target person without having a special device attached to the target person.

(Item 7) The health evaluation system according to any one of items 1 to 6, in which the acquisition unit acquires the blood pressure value of the target person based on an image of the target person from an outside camera that is provided in the moving body and acquires an image of surroundings of the moving body.

According to the health evaluation system of item 7, it is possible to have any person in the surroundings of the moving body as the target person and acquire the blood pressure value of the target person without having a special device attached to the target person.

(Item 8) The health evaluation system according to any one of items 1 to 7, in which the acquisition unit acquires the blood pressure value of the target person by: receiving a blood pressure value measured by a wearable terminal attached to a body of the target person from the wearable terminal; or receiving biological data measured by the wearable terminal, and calculating the blood pressure value based on the received biological data.

According to the health evaluation system of item 8, the blood pressure value is acquired by using the wearable terminal that is attached to the body of the target person and capable of acquiring the biological data in the immediate vicinity of the body, so that it is possible to increase the measurement precision of the blood pressure value and evaluate the health condition more accurately.

(Item 9) The health evaluation system according to any one of items 1 to 8, in which the evaluation unit evaluates the health condition of the target person based on: the standing-state blood pressure value that is the blood pressure value measured when the target person is heading toward the moving body by legged locomotion that is locomotion using own legs in a standing state; and the seated-state blood pressure value that is the blood pressure value of the target person measured after boarding the moving body and seated on the seat.

According to the health evaluation system of item 9, changes in the blood pressure due to changes in the exercise load can be grasped explicitly, so that it is possible to more accurately evaluate the health condition of the target person boarding the moving body.

(Item 10) A health evaluation method executed by a computer, the health evaluation method including: an acquisition step of acquiring a blood pressure value of a target person; and an evaluation step of evaluating a health condition of the target person based on the blood pressure value of the target person acquired in the acquisition step, in which the evaluation step evaluates the health condition of the target person based on: a standing-state blood pressure value that is a blood pressure value measured when the target person is heading toward the moving body by legged locomotion that is locomotion using own legs in a standing state; and a seated-state blood pressure value that is a blood pressure value of the target person measured after boarding the moving body and seated on a seat.

According to the health evaluation method of item 10, the health condition of the target person is evaluated based on the blood pressure value under a standing state measured when the target person is actually onboard the moving body and the blood pressure value under a seated state, so that it is possible to appropriately support the target person in regards to traveling in the moving body based on the evaluation result.

(Item 11) A health evaluation program causing a computer of a health evaluation system to function as: an acquisition unit that acquires a blood pressure value of a target person; and an evaluation unit that evaluates a health condition of the target person based on the blood pressure value of the target person acquired by the acquisition unit, in which the evaluation unit evaluates the health condition of the target person based on: a standing-state blood pressure value that is a blood pressure value measured when the target person is heading toward a moving body in a standing state; and a seated-state blood pressure value that is a blood pressure value of the target person measured after boarding the moving body and seated on a seat.

According to the health evaluation program of item 11, it is possible to implement the health evaluation system that is capable of appropriately evaluating the health condition of the target person when onboard the moving body and appropriately supporting the target person in regards to traveling in the moving body.

### REFERENCE SIGNS LIST

1 Health evaluation system
2 Vehicle
3 Wearable terminal
4, 9 Mobile terminal
5 Evaluation support device
7 Communication network
8 Server
10 Driver's seat
11 Instrument panel
12 AV device
13 First touch panel
14 Inside camera
15 Speaker
16 Microphone
17 Door mirror
18 Outside camera
19 Seating sensor
20 First processor
21 First memory
22 First communication device
23 Second touch panel
24 First GNSS sensor
25 Acceleration sensor
30 Legged locomotion detection unit
31 Approach detection unit
32 Seating detection unit
33 Acquisition unit
34 Evaluation unit
35 Notification unit
36 Information transmission unit
39 First program
50 Second processor
51 Second memory
52 Second communication device
53 Second GNSS sensor
60 Communication control unit
61 Image providing unit
62 Seating monitoring unit
63 AV control unit
64 Positional information transmission unit
69 Second program

What is claimed is:

1. A health evaluation system comprising:
an acquisition unit that acquires a blood pressure value of a target person; and
an evaluation unit that evaluates a health condition of the target person based on the blood pressure value of the target person acquired by the acquisition unit, wherein
the evaluation unit evaluates the health condition of the target person based on: a standing-state blood pressure value that is a blood pressure value measured when the target person is heading toward a moving body in a standing state; and a seated-state blood pressure value that is a blood pressure value of the target person measured after boarding the moving body and seated on a seat.

2. The health evaluation system according to claim 1, wherein the evaluation unit takes, as the standing-state blood pressure value, a blood pressure value acquired by the acquisition unit when the target person is within a predetermined distance range from the moving body.

3. The health evaluation system according to claim 1, wherein the evaluation unit takes:
as the standing-state blood pressure value, a blood pressure value acquired immediately after the target person in a standing state boards the moving body and is seated on the seat of the moving body; and
as the seated-state blood pressure value, a blood pressure value acquired after a prescribed time defined in advance has passed after the target person is seated on the seat of the moving body.

4. The health evaluation system according to claim 1, comprising a notification unit that issues a notification based on an evaluation result regarding the health condition of the target person evaluated in the evaluation unit, wherein
the notification unit determines whether to issue the notification, based on the evaluation result regarding the health condition of the target person.

5. The health evaluation system according to claim 4, comprising an information transmission unit that transmits positional information regarding a current position of the moving body to an external server of the moving body at prescribed time intervals, wherein
the information transmission unit determines whether to transmit the positional information to the server based on the evaluation result regarding the health condition of the target person evaluated in the evaluation unit, or based on whether the notification is issued by the notification unit.

6. The health evaluation system according to claim 1, wherein the acquisition unit acquires the blood pressure value of the target person based on an image of the target person from an inside camera that is provided in the moving body and acquires an image of inside of the moving body.

7. The health evaluation system according to claim 1, wherein the acquisition unit acquires the blood pressure value of the target person based on an image of the target person from an outside camera that is provided in the moving body and acquires an image of surroundings of the moving body.

8. The health evaluation system according to claim 1, wherein the acquisition unit acquires the blood pressure value of the target person by: receiving a blood pressure value measured by a wearable terminal attached to a body of the target person from the wearable terminal; or receiving biological data measured by the wearable terminal, and calculating the blood pressure value based on the received biological data.

9. The health evaluation system according to claim 1, wherein the evaluation unit evaluates the health condition of the target person based on: the standing-state blood pressure value that is the blood pressure value measured when the target person is heading toward the moving body by legged locomotion that is locomotion using own legs in a standing state; and the seated-state blood pressure value that is the blood pressure value of the target person measured after boarding the moving body and seated on the seat.

10. A health evaluation method executed by a computer, the health evaluation method comprising:
an acquisition step of acquiring a blood pressure value of a target person; and
an evaluation step of evaluating a health condition of the target person based on the blood pressure value of the target person acquired in the acquisition step, wherein
the evaluation step evaluates the health condition of the target person based on: a standing-state blood pressure value that is a blood pressure value measured when the target person is heading toward the moving body by legged locomotion that is locomotion using own legs in a standing state; and a seated-state blood pressure value that is a blood pressure value of the target person measured after boarding the moving body and seated on a seat.

11. A non-transitory computer-readable storage medium storing a health evaluation program which is executed by a computer of a health evaluation system, wherein the health evaluation program makes the computer functions as:
an acquisition unit that acquires a blood pressure value of a target person; and
an evaluation unit that evaluates a health condition of the target person based on the blood pressure value of the target person acquired by the acquisition unit, wherein
the evaluation unit evaluates the health condition of the target person based on: a standing-state blood pressure value that is a blood pressure value measured when the target person is heading toward a moving body in a standing state; and a seated-state blood pressure value that is a blood pressure value of the target person measured after boarding the moving body and seated on a seat.
